(19) United States
(12) Patent Application Publication
(10) Pub. No.: US 2003/0140362 A1
(43) Pub. Date: Jul. 24, 2003

(54) In vivo models for screening inhibitors of hepatitis B virus

(76) Inventors: Dennis Macejak, Arvada, CO (US); Patrice Lee, Erie, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/279,401

(22) Filed: Oct. 24, 2002

Related U.S. Application Data
(63) Continuation-in-part of application No. PCT/US02/09187, filed on Mar. 26, 2002.
(60) Provisional application No. 60/296,876, filed on Jun. 8, 2001.
(60) Provisional application No. 60/335,059, filed on Oct. 24, 2001.

(51) Int. Cl. A01K 67/027; G01N 33/00; C12Q 1/70

(52) U.S. Cl. 800/18; 800/21; 435/5

(57) ABSTRACT

The present invention relates to compounds, compositions, and methods for the study, diagnosis, and treatment of disease states related to hepatitis B virus (HBV) replication and gene expression. HBV animal models and methods of use are provided, including methods of screening for compounds and/or potential therapies directed against HBV.

### RELATED APPLICATIONS

This patent application is a continuation-in-part of PCT/US02/09187, which has not yet been published, which claims priority from both Macejak et al., U.S. Ser. No. 60/296,876, filed Jun. 8, 2001 and U.S. Ser. No. 60/335,059 filed Oct. 24, 2001, which are incorporated by reference herein in their entirety including the drawings.

### BACKGROUND OF THE INVENTION

The present invention relates to compounds, compositions, and methods for the study, diagnosis, and treatment of disease states related to hepatitis B virus (HBV) replication and gene expression. Specifically, the invention relates to models and systems for screening inhibitors of HBV replication and propagation.

The following is a discussion of relevant art pertaining to hepatitis B virus (HBV). The discussion is not meant to be complete and is provided only for understanding of the invention that follows. The summary is not an admission that any of the work described below is prior art to the claimed invention.

Chronic hepatitis B is caused by an enveloped virus, commonly known as the hepatitis B virus or HBV. HBV is transmitted via infected blood or other body fluids, especially saliva and semen, during delivery, sexual activity, or sharing of needles contaminated by infected blood. Individuals can be “carriers” and transmit the infection to others without ever having experienced symptoms of the disease. Persons at highest risk are those with multiple sex partners, those with a history of sexually transmitted diseases, parenteral drug users, infants born to infected mothers, “close” contacts or sexual partners of infected persons, and healthcare personnel or other service employees who have contact with blood. Transmission is also possible via tattooing, ear or body piercing, and acupuncture; the virus is also stable on razors, toothbrushes, baby bottles, eating utensils, and some hospital equipment such as respirators, scopes and instruments. There is no evidence that HbsAg (HBV surface antigen) positive food handlers pose a health risk in an occupational setting, hence, they should not be excluded from the workplace. Hepatitis B has never been documented as being a food-borne disease. The average incubation period is 60 to 90 days, with a range of 45 to 180; the number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of severity include: (1) The size of the dose to which the person was exposed; (2) the person's age with younger patients experiencing a milder form of the disease; (3) the status of the immune system with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and tiredness/loss of energy. Jaundice is not experienced in all cases, however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement can occur accompanied by right upper-quadrant pain. The acute phase of the disease can be accompanied by severe depression, meningitis, Guillain-Barré syndrome, myelitis, encephalitis, agranulocytosis, and/or thrombocytopenia.

Hepatitis B is generally self-limiting and will resolve in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life become carriers.

Hepatitis B virus (HBV) infects over 300 million people worldwide (Imperial, 1999, Gastroenterol. Hepatol., 14 (suppl), S1-5). In the United States approximately 1.25 million individuals are chronic carriers of HBV as evidenced by the fact that they have measurable hepatitis B virus surface antigen HBsAg in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation is the only treatment option for patients with end-stage liver disease from HBV.

The natural progression of chronic HBV infection over a 10 to 20 year period leads to cirrhosis in 20-to-50% of patients and progression of HBV infection to hepatocellular carcinoma has been well documented. There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., 1993, American Journal of Gastroenterology, 88, 240-243). Treatment of hepatocellular carcinoma with chemotherapeutic agents has not proven effective and only 10% of patients will benefit from surgery due to extensive tumor invasion of the liver (Trinchet et al., 1994,Presse Medicine, 23, 831-833). Given the aggressive nature of primary hepatocellular carcinoma, the only viable treatment alternative to surgery is liver transplantation (Pichlmayr et al., 1994, Hepatology., 20, 33S-40S).

Upon progression to cirrhosis, patients with chronic HCV infection present with clinical features, which are common to clinical cirrhosis regardless of the initial cause (D'Amico et al., 1986, Digestive Diseases and Sciences, 31, 468-475). These clinical features can include: bleeding esophageal varices, ascites, jaundice, and encephalopathy (Zakim D, Boyer T D. Hepatology a textbook of liver disease, Second Edition Volume 1. 1990 W. B. Saunders Company. Philadelphia). In the early stages of cirrhosis, patients are classified as compensated, meaning that although liver tissue damage has occurred, the patient's liver is still able to detoxify metabolites in the bloodstream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding, and 16% had encephalopathy. Hepatocellular carcinoma was observed in 6 (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six year study.

With respect to survival, the D'Amico study indicated that the five year survival rate for all patients on the study was only 40%. The six year survival rate for the patients who initially had compensated cirrhosis was 54% while the six year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Hepatitis B virus is a double-stranded circular DNA virus. It is a member of the Hepadnaviridae family. The virus is 42 nm in diameter, consisting of a central core that contains a core antigen (HBcAg) surrounded by an envelope containing a surface protein/surface antigen (HBsAg). It also contains an e antigen (HBeAg) that, along with HBcAg and HBsAg, is helpful in identifying this disease.

In HBV virions, the genome is found in an incomplete double-stranded form. HBV uses a reverse transcriptase to transcribe a positive-sense full length RNA version of its genome back into DNA. This reverse transcriptase also contains DNA polymerase activity, and thus, begins replicating the newly synthesized minus-sense DNA strand. However, it appears that the core protein encapsidates the reverse-transcriptase/polymerase before it completes replication.

From the free-floating form, the virus must first attach itself specifically to a host cell membrane. Viral attachment is one of the crucial steps that determine host and tissue specificity. Currently there are no in vitro cell lines that can be infected by HBV. There are, however, some cell lines, such as HepG2, which can support viral replication only upon transient or stable transfection using HBV DNA.

Cell Culture Models

As previously mentioned HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA can be co-transfected with ribozymes in cell culture. Such an approach has been used to report intracellular ribozyme activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV. In such cells, only the delivery of ribozymes is required; however, a delivery screen must be performed.

Phenotypic Assays

Intracellular HBV gene expression can be assayed either by a Taqman® assay for HBV RNA or by ELISA for HBV protein. Extracellular virus can be assayed either by PCR for DNA or ELISA for protein. Antibodies are commercially available for HBV surface antigen and core protein. A secreted alkaline phosphatase expression plasmid can be used to normalize for differences in transfection efficiency and sample recovery.

Animal Models

There are several small animal models used to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

Therapeutic Approaches

Current therapeutic goals of treatment are three-fold: to eliminate infectivity and transmission of HBV to others, to arrest the progression of liver disease and improve the clinical prognosis, and to prevent the development of hepatocellular carcinoma (HCC).

Interferon alpha is the most common therapeutic for HBV; however, the FDA has recently approved Lamivudine (3TC®) as a therapeutic. Interferon alpha (IFN-alpha) is one treatment for chronic hepatitis B. The standard duration of IFN-alpha therapy is 16 weeks, however, the optimal treatment length is still poorly defined. A complete response (HBV DNA negative HBeAg negative) occurs in approximately 25% of patients. Several factors have been identified that predict a favorable response to therapy including: high ALT, low HBV DNA , being female, and heterosexual orientation.

There is also a risk of reactivation of the hepatitis B virus even after a successful response, this occurs in around 5% of responders and normally occurs within 1 year.

Side effects resulting from treatment with type 1 interferons can be divided into four general categories including: influenza-like symptoms, neuropsychiatric, laboratory abnormalities, and other miscellaneous side effects. Examples of influenza-like symptoms include, fatigue, fever; myalgia, malaise, appetite loss, tachycardia, rigors, headache and arthralgias. The influenza-like symptoms are usually short-lived and tend to abate after the first four weeks of dosing (Dusheiko et al., 1994, Journal of Viral Hepatitis, 1, 3-5). Neuropsychiatric side effects include irritability, apathy, mood changes, insomnia, cognitive changes, and depression. Laboratory abnormalities include the reduction of myeloid cells, including granulocytes, platelets and to a lesser extent, red blood cells. These changes in blood cell counts rarely lead to any significant clinical sequellae. In addition, increases in triglyceride concentrations and elevations in serum alaine and aspartate aminotransferase concentration have been observed. Finally, thyroid abnormalities have been reported. These thyroid abnormalities are usually reversible after cessation of interferon therapy and can be controlled with appropriate medication during therapy. Miscellaneous side effects include nausea, diarrhea, abdominal and back pain, pruritus, alopecia, and rhinorrhea. In general, most side effects will abate after 4 to 8 weeks of therapy (Dushieko et al., supra ).

Lamivudine (3TC®) is a nucleoside analogue, which is a very potent and specific inhibitor of HBV DNA synthesis. Lamivudine has recently been approved for the treatment of chronic Hepatitis B. Unlike treatment with interferon, treatment with 3TC® does not eliminate the HBV from the patient. Rather, viral replication is controlled and chronic administration results in improvements in liver histology in over 50% of patients. Phase III studies with 3TC®, showed that treatment for one year was associated with reduced liver inflammation and a delay in scarring of the liver. In addition, patients treated with Lamivudine (100 mg per day) had a 98% reduction in hepatitis B DNA and a significantly higher rate of seroconversion, suggesting disease improvements after completion of therapy. However, cessation of therapy resulted in a reactivation of HBV replication in most patients. In addition, recent reports have documented 3TC® resistance in approximately 30% of patients.

Current therapies for treating HBV infection, including interferon and nucleoside analogues, are only partially effective. In addition, drug resistance to nucleoside analogues is now emerging, making treatment of chronic Hepatitis B more difficult. Thus, a need exists for effective treatment of this disease that utilizes antiviral inhibitors that work by mechanisms other than those currently utilized in the treatment of both acute and chronic hepatitis B infections.

### SUMMARY OF THE INVENTION

The invention relates to in vitro and in vivo systems for screening inhibitors of HBV. In one embodiment, the invention features a mouse, for example a male or female mouse, implanted with HepG2.2.15 cells, wherein the mouse is susceptible to HBV infection and capable of sustaining HBV DNA expression. One embodiment of the invention provides a mouse implanted with HepG2.2.15 cells, wherein said mouse sustains the propagation of HEPG2.2.15 cells and HBV production.

In another embodiment, a mouse of the invention has been infected with HBV for at least one week to at least eight weeks, including, for example at least 4 weeks.

In yet another embodiment, a mouse of the invention, for example a male or female mouse, is an immunocompromised mouse, for example a nu/nu mouse or a scid/scid mouse.

In one embodiment, the invention features a method of producing a mouse of the invention, comprising injecting, for example by subcutaneous injection, HepG2.2.15 (Sells, et al,. 1987, Proc Natl Acad Sci U S A., 84, 1005-1009) cells into the mouse under conditions suitable for the propagation of HepG2.2.15 cells in said mouse. HepG2.2.15 cells can be suspended in, for example, Dulbecco's PBS solution including calcium and magnesium. In another embodiment, HepG2.2.15 cells are selected for antibiotic resistance and are then introduced into the mouse under conditions suitable for the propagation of HepG2.2.15 cells in said mouse. A non-limiting example of antibiotic resistant HepG2.2.15 cells include G418 antibiotic resistant HepG2.2.15 cells.

In another embodiment, the invention features a method for screening a potential therapeutic compound or compounds for activity against HBV, comprising administering the compound to a mouse of the invention and monitoring the levels of HBV in the mouse to determine anti-HBV activity On way to determine the level of HBV is to, for example, assay for HBV DNA levels

A compound(s) or potential therapy contemplated by the invention includes, for example, a nucleic acid molecule, lipid, steroid, peptide, protein, antibody, monoclonal antibody, humanized monoclonal antibody, small molecule, and/or isomers and analogs thereof, and/or a cell.

In one embodiment, a compound or therapy contemplated by the invention is a nucleic acid molecule, such as an enzymatic nucleic acid molecule, antisense nucleic acid molecule, allozyme, peptide nucleic acid, decoy, triplex oligonucleotide, dsRNA, ssRNA, RNAi, siRNA, aptamer, or 2,5-A chimera used alone or in combination with another compound or therapy, for example, antiviral therapy. Antiviral compounds and therapy can be, for example, treatment with 3TC® (Lamivudine) or interferon. Interferon can include, for example, consensus interferon or type I interferon. Type I interferon can include interferon alpha, interferon beta, consensus interferon, polyethylene glycol interferon, polyethylene glycol interferon alpha 2a, polyethylene glycol interferon alpha 2b, or polyethylene glycol consensus interferon.

The above-described screening method can be used to screen one or more compounds, for example, any of the compounds described herein or any combination thereof, for anti-HBV activity. For example, in one embodiment, the screening method can be used to screen one or more nucleic acid molecules. In another embodiment, the screening method can be used to screen a nuleic acid molecule and an antiviral compound, such as, for example, interferon and Lamivudine.

In one embodiment, the invention features a non-human mammal implanted with HepG2.2.15 cells, wherein the non-human mammal is susceptible to HBV infection and capable of sustaining HBV DNA expression in the HepG2.2.15 cells implanted.

In another embodiment, a non-human mammal of the invention, for example a male or female non-human mammal, has been infected with HBV for at least one week to at least eight weeks, including for example at least four weeks.

In yet another embodiment, a non-human mammal of the invention is an immunocompromised mammal, for example a nu/nu mammal or a scid/scid mammal.

In one embodiment, the invention features a method of producing a non-human mammal of the invention, comprising injecting, for example by subcutaneous injection, HepG2.2.15 cells into the non-human mammal, under conditions suitable for the propagation of HepG2.2.15 cells in said non-human mammal.

In another embodiment, the invention features a method for screening a potential therapeutic compound or compounds for activity against HBV, comprising administering the compound to a non human mammal of the invention and monitoring the levels of HBV in the mouse to determine anti-HBV activity On way to determine the level of HBV is to, for example, assay for HBV DNA levels

In one embodiment, a compound or therapy contemplated by the invention is a nucleic acid molecule, for example an enzymatic nucleic acid molecule, allozyme, antisense nucleic acid molecule, decoy, triplex oligonucleotide, dsRNA, ssRNA, RNAi, siRNA, or 2,5-A chimera used alone or in combination with another compound or therapy, for example an antiviral compound or therapy such as those described herein and known in the art.

The above-described screening method can be used to screen one or more compounds, for example, any of the compounds described herein or any combination thereof, for anti-HBV activity. For example, in one embodiment, the screening method can be used to screen one or more nucleic acid molecules. In another embodiment, the screening method can be used to screen a nuleic acid molecule and an antiviral compound, such as, for example, interferon and Lamivudine.

Methods and chimeric immunocompromised heterologous non-human mammalian hosts, particularly mouse hosts, are provided for the expression of hepatitis B virus (“HBV”). The chimeric hosts have transplanted viable, HepG2.2.15 cells in an immunocompromised host.

The non-human mammals are immunocompromised in normally inheriting the desired immune incapacity, or the desired immune incapacity can be created. For example, hosts with severe combined immunodeficiency, known as scid/scid hosts, are available. Rodentia, particularly mice, and equine, particularly horses, are presently available as scid/scid hosts, for example scid/scid mice and scid/scid rats. The scid/scid hosts lack functioning lymphocyte types, particularly B-cells and some T-cell types. In the scid/scid mouse hosts, the genetic defect appears to be a non-functioning recombinase, as the germline DNA is not rearranged to produce functioning surface immunoglobulin and T-cell receptors.

Any immunodeficient mouse can be used to generate the animal models described herein. The term “immunodeficient,” as used herein, refers to a genetic alteration that impairs the animal's ability to mount an effective immune response. In this regard, an “effective immune response” is one which is capable of destroying invading pathogens such as (but not limited to) viruses, bacteria, parasites, malignant cells, and/or a xenogeneic or allogeneic transplant. In one embodiment, the immunodeficient mouse is a severe immunodeficient (SCID) mouse, which lacks recombinase activity that is necessary for the generation of immunoglobulin and functional T cell antigen receptors, and thus does not produce functional B and T lymphocytes. In another embodiment, the immunodeficient mouse is a nude mouse, which contains a genetic defect that results in the absence of a functional thymus, leading to T-cell and B-cell deficiencies. However, mice containing other immunodeficiencies (such as rag-1 or rag-2 knockouts, as described in Chen et al., 1994, Curr. Opin. Immunol., 6, 313-319 and Guidas et al., 1995, J. Exp. Med., 181, 1187-1195, or beige-nude mice, which also lack natural killer cells, as described in Kollmann et al., 1993, J. Exp. Med., 177, 821-832) can also be employed.

The introduction of HepG2.2.15 cells occurs with a host at an age less than about 25% of its normal lifespan, usually to 20% of the normal lifespan with mice, and the age is generally about 3 to 10 weeks, more usually from about 4 to 8 weeks. The mice can be of either sex, can be neutered, and can be otherwise normal, except for the immunocompromised state, or they can have one or more mutations, which can be naturally occurring or as a result of mutagenesis.

In another embodiment, the above mouse model is used to evaluate the effectiveness of therapies. The term “therapy,” as used herein, encompasses exogenous factors, such as dietary or environmental conditions, as well as pharmaceutical compositions “drugs” and vaccines. In one embodiment, the therapy is an immunotherapy, which can include the treatment of the HBV bearing animal with populations of HBV-reactive immune cells. The therapy can also, or alternatively, be a gene therapy (i.e., a therapy that involves treatment of the HBV-bearing mouse with a cell population that has been manipulated to express one or more genes, the products of which can possess anti-viral activity), see for example The Development of Human Gene Therapy, Theodore Friedmann, Ed. Cold Spring Harbor Laboratory Press, Cold Spring Harbor, N.Y., 1999. Therapies of the invention can also comprise a drug or composition with pharmaceutical activity that can be used to treat illness or disease. A therapy can comprise a plurality of compounds in a mixture or a distinct entity. Examples of such compounds include nucleosides, nucleic acids, nucleic acid chimeras, RNA and DNA oligonucleotides, peptide nucleic acids, enzymatic nucleic acid molecules, antisense nucleic acid molecules, decoys, triplex oligonucleotides, ssDNA, dsRNA, ssRNA, siRNA, 2,5-A chimeras, lipids, steroids, peptides, proteins, antibodies, monoclonal antibodies (see for example Hall, 1995, Science, 270, 915-916), small molecules, and/or isomers and analogs thereof.

In another embodiment, the invention features novel nucleic acid-based techniques such as enzymatic nucleic acid molecules, antisense molecules, and methods for their use to down-regulate or inhibit the expression of HBV RNA and/or replication of HBV.

In yet another embodiment, the invention features the use of one or more of the enzymatic nucleic acid-based techniques to inhibit the expression of the genes encoding HBV viral proteins. Specifically, the invention features the use of enzymatic nucleic acid-based techniques to specifically inhibit the expression of the HBV viral genome.

In one embodiment, the invention features nucleic acid-based inhibitors (e.g., enzymatic nucleic acid molecules, antisense nucleic acids, triplex DNA, antisense nucleic acids containing RNA cleaving chemical groups) and methods for their use to down-regulate or inhibit the expression of RNA (e.g., HBV) capable of progression and/or maintenance of hepatitis, hepatocellular carcinoma, cirrhosis, and/or liver failure.

In one embodiment, the compounds of the invention are used to treat a HBV infected patient wherein the HBV is resistant or the patient does not respond to treatment with 3TC® (Lamivudine), either alone or in combination with other therapies under conditions suitable for the treatment.

In another embodiment, the compounds of the invention are used to treat HBV infected cells or a HBV infected patient wherein the HBV is resistant or the patient does not respond to treatment with interferon, for example Infergen®, either alone or in combination with other therapies under conditions suitable for the treatment.

Nucleic acid molecules of the invention exhibit a high degree of specificity for only the viral mRNA in infected cells. Nucleic acid molecules of the instant invention targeted to highly conserved sequence regions allow the treatment of many strains of human HBV with a single compound. No treatment presently exists which specifically attacks expression of the viral gene(s) that are responsible for transformation of hepatocytes by HBV.

The compounds, therapies and methods of this invention can be used to treat human hepatitis B virus infections, which include productive virus infection, latent or persistent virus infection, and HBV-induced hepatocyte transformation. The utility can be extended to other species of HBV that infect non-human animals where such infections are of veterinary importance.

Target sites of nucleic acid molecules of the invention comprise genes required for viral replication. A non-limiting example of such targets include genes for protein synthesis, such as the 5′ most 1500 nucleotides of the HBV pregenomic mRNAs. For sequence references, see Renbao et al., 1987, Sci. Sin., 30, 507. This region controls the translational expression of the core protein (C), X protein (X) and DNA polymerase (P) genes and plays a role in the replication of the viral DNA by serving as a template for reverse transcriptase. Disruption of this region in the RNA results in deficient protein synthesis as well as incomplete DNA synthesis (and inhibition of transcription from the defective genomes). Target sequences 5′ of the encapsidation site can result in the inclusion of the disrupted 3′ RNA within the core virion structure and targeting sequences 3′ of the encapsidation site can result in the reduction in protein expression from both the 3′ and 5′ fragments.

Alternative regions outside of the 5′ most 1500 nucleotides of the pregenomic mRNA also provide targets of enzymatic nucleic acid mediated inhibition of HBV replication. Such targets include the mRNA regions that encode the viral S gene. Selection of particular target regions will depend upon the secondary structure of the pregenomic mRNA. Targets in the minor mRNAs can also be used, especially when folding or accessibility assays in these other RNAs reveal additional target sequences that are unavailable in the pregenomic mRNA species.

A target in the pregenomic RNA is a proposed bipartite stem-loop structure in the 3′-end of the pregenomic RNA which is believed to be critical for viral replication (Kidd and Kidd-Ljunggren, 1996. Nuc. Acid Res. 24:3295-3302). The 5′-end of the HBV pregenomic RNA carries a cis-acting encapsidation signal, which has inverted repeat sequences that are thought to form a bipartite stem-loop structure. Due to a terminal redundancy in the pregenomic RNA, the putative stem-loop also occurs at the 3′-end. While it is the 5′ copy which functions in polymerase binding and encapsidation, reverse transcription actually begins from the 3′ stem-loop. To start reverse transcription, a 4 nt primer which is covalently attached to the polymerase is made, using a bulge in the 5′ encapsidation signal as template. This primer is then shifted, by an unknown mechanism, to the DR1 primer binding site in the 3′ stem-loop structure, and reverse transcription proceeds from that point. The 3′ stem-loop, and especially the DR1 primer binding site, appear to be highly effective targets for nucleic acid intervention.

Sequences of the pregenomic RNA are shared by the mRNAs for surface, core, polymerase, and X proteins. Due to the overlapping nature of the HBV transcripts, all share a common 3′-end. Nucleic acid targeting of this common 3′-end can be used to modulate the expression of pregenomic RNA as well as all of the mRNAs for surface, core, polymerase, and X proteins.

In one embodiment, the invention features the use of an enzymatic nucleic acid molecule, preferably in the hammerhead, NCH, G-cleaver, amberzyme, zinzyme and/or DNAzyme motif, to down-regulate the expression of the HBV genome.

The term “inhibit” or “down-regulate” as used herein refers to the expression of the gene, or level of RNAs or equivalent RNAs encoding one or more protein subunits or components, or activity of one or more protein subunits or components, such as HBV protein or proteins, is reduced below that observed in the absence of the therapies of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of HBV with the nucleic acid molecule of the instant invention is greater in the presence of the nucleic acid molecule than in its absence.

The term “up-regulate” as used herein refers to the expression of the gene, or level of RNAs or equivalent RNAs encoding one or more protein subunits or components, or activity of one or more protein subunits or components, such as HBV protein or proteins, is greater than that observed in the absence of the therapies of the invention. For example, the expression of a gene, such as HBV genes, can be increased in order to treat, prevent, ameliorate, or modulate a pathological condition caused or exacerbated by an absence or low level of gene expression.

The term “modulate” as used herein refers to the expression of the gene, or level of RNAs or equivalent RNAs encoding one or more protein subunits or components, or activity of one or more proteins is up-regulated or down-regulated, such that the expression, level, or activity is greater than or less than that observed in the absence of the therapies of the invention.

The term “enzymatic nucleic acid molecule” as used herein refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

The term “nucleic acid molecule” as used herein refers to a molecule having nucleotides. The nucleic acid can be single, double, or multiple stranded and can comprise modified or unmodified nucleotides or non-nucleotides or various mixtures and combinations thereof.

The term “enzymatic portion” or “catalytic domain” as used herein refers to that portion/region of the enzymatic nucleic acid molecule essential for cleavage of a nucleic acid substrate (for example see FIGS. 1-4).

The term “substrate binding arm” or “substrate binding domain” as used herein refers to that portion/region of an enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

The term “Inozyme” or “NCH” motif or configuration as used herein refers to an enzymatic nucleic acid molecule comprising a motif as is generally described as NCH Rz in FIG. 1. Inozymes possess endonuclease activity to cleave RNA substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave RNA substrates having a cleavage triplet NCN/, where N is a nucleotide, C is cytidine, and “/” represents the cleavage site. “I” in FIG. 1 represents an Inosine nucleotide, preferably a ribo-Inosine or xylo-Inosine nucleoside.

The term “G-cleaver” motif or configuration as used herein refers to an enzymatic nucleic acid molecule comprising a motif as is generally described as G-cleaver Rz in FIG. 1. G-cleavers possess endonuclease activity to cleave RNA substrates having a cleavage triplet NYN/, where N is a nucleotide, Y is uridine or cytidine and “/” represents the cleavage site. G-cleavers can be chemically modified as is generally shown in FIG. 1.

The term “amberzyme” motif or configuration as used herein refers to an enzymatic nucleic acid molecule comprising a motif as is generally described in FIG. 2. Amberzymes possess endonuclease activity to cleave RNA substrates having a cleavage triplet NG/N, where N is a nucleotide, G is guanosine, and “I” represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

The term “zinzyme” motif or configuration as used herein refers to an enzymatic nucleic acid molecule comprising a motif as is generally described in FIG. 3. Zinzymes possess endonuclease activity to cleave RNA substrates having a cleavage triplet including but not limited to YG/Y, where Y is uridine or cytidine, and G is guanosine and “/” represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

The term ‘DNAzyme’ as used herein refers to an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection (see Santoro et al., supra). Additional DNAzyme motifs can be selected for using techniques similar to those described in these references, and hence, are within the scope of the present invention.

The term “sufficient length” as used herein refers to an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

The term “stably interact” as used herein refers to the interaction of oligonucleotides with target nucleic acid molecules (e.g., by forming hydrogen bonds with complementary nucleotides in the target under physiological conditions) that is sufficient to the intended purpose (e.g., cleavage of target RNA by an enzyme).

The term “equivalent” RNA to HBV is meant to include those naturally occurring RNA molecules having homology (partial or complete) to HBV proteins or encoding for proteins with similar function as HBV proteins in various organisms, including humans, rodents, primates, rabbits, pigs, protozoans, fungi, plants, and other microorganisms and parasites. The equivalent RNA sequence also includes, in addition to the coding region, regions such as a 5′-untranslated region, a 3′-untranslated region, introns, a intron-exon junction and the like.

The term “homology” as used herein refers to the nucleotide sequence of two or more nucleic acid molecules is partially or completely identical.

The term “component” of HBV as used herein refers to a peptide or protein subunit expressed from a HBV gene.

The term “antisense nucleic acid”, as used herein refers to a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

The term “RNase H activating region” as used herein refers to a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). An RNase H enzyme binds to a nucleic acid molecule-target RNA complex and cleaves the target RNA sequence. An RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, an RNase H activating region can also comprise a variety of sugar chemistries. For example, an RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of an RNase H activating region and the instant invention.

The term “single stranded RNA” (ssRNA) as used herein refers to a naturally occurring or synthetic ribonucleic acid molecule comprising a linear single strand, for example a ssRNA can be a messenger RNA (mRNA), transfer RNA (tRNA), ribosomal RNA (rRNA) etc. of a gene.

The term “single stranded DNA” (ssDNA) as used herein refers to a naturally occurring or synthetic deoxyribonucleic acid molecule comprising a linear single strand, for example, a ssDNA can be a sense or antisense gene sequence or EST (Expressed Sequence Tag).

The term “double stranded RNA” or “dsRNA” as used herein refers to a double stranded RNA molecule capable of RNA interference, including short interfering RNA (siRNA), see for example Bass, 2001, Nature, 411, 428-429; Elbashir et al., 2001, Nature, 411, 494-498)

The term “allozyme” as used herein refers to an allosteric enzymatic nucleic acid molecule, see for example see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.The term “2-5A chimera” as used herein refers to an oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “triplex forming oligonucleotides” as used herein refers to an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

The term “gene” as used herein refers to a nucleic acid that encodes an RNA, for example, nucleic acid sequences including but not limited to structural genes encoding a polypeptide.

The term “complementarity” as used herein refers to the ability of a nucleic acid to form hydrogen bond or bonds with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The term “RNA” as used herein refers to a molecule comprising at least one ribonucleotide residue. By “ribonucleotide” or “2′-OH” is meant a nucleotide with a hydroxyl group at the 2′ position of a β-D-ribo-furanose moiety.

The term “decoy ” as used herein refers to a nucleic acid molecule, for example RNA or DNA, or aptamer that is designed to preferentially bind to a predetermined ligand. Such binding can result in the inhibition or activation of a target molecule. A decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy can be designed to bind to HBV proteins and block the binding of HBV DNA or RNA or a decoy can be designed to bind to HBV proteins and prevent molecular interaction with the HBV proteins.

Several varieties of naturally occurring enzymatic RNAs are known presently. Each can catalyze the hydrolysis of RNA phosphodiester bonds in trans (and thus can cleave other RNA molecules) under physiological conditions. Table I summarizes some of the characteristics of these enzymatic nucleic acid molecules. In general, enzymatic nucleic acids act by first binding to a target RNA. Such binding occurs through the target binding portion of a enzymatic nucleic acid that is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid molecule is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a enzymatic nucleic acid molecule.

Enzymatic nucleic acid molecules that cleave specified sites in HBV-specific RNAs represent a therapeutic approach to treat HBV infection, hepatocellular carcinoma, or any other disease or condition that responds to the modulation of HBV expression.

In one embodiment of the inventions described herein, an enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of hepatitis delta virus motifs by Perrotta and Been, 1992 Biochemistry 31, 16; of RNase P motifs by Guerrier-Takada et al., 1983 Cell 35, 849; Forster and Altman, 1990, Science 249, 783; Li and Altman, 1996, Nucleic Acids Res. 24, 835; of Neurospora VS RNA ribozyme motifs by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); of Group II introns by Griffin et al., 1995, Chem. Biol. 2, 761; Michels and Pyle, 1995, Biochemistry 34, 2965; Pyle et al., International PCT Publication No. WO 96/22689; of Group I introns by Cech et al., U.S. Pat. No. 4,987,071, and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site that is complementary to one or more target gene RNA regions, and that it have nucleotide sequences within or surrounding a substrate binding site that impart an RNA cleaving activity to the molecule (Cech et al., U.S. Pat. No. 4,987,071).

In one embodiment of the present invention, a nucleic acid molecule of the instant invention can be between about 10 and 100 nucleotides in length. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for a nucleic acid molecule to be of length and conformation sufficient and suitable for the nucleic acid molecule to interact with its target and/or catalyze a reaction contemplated herein. The length of nucleic acid molecules of the instant invention are not limiting within the general limits stated.

Preferably, a nucleic acid molecule that modulates, for example, down-regulates HBV expression, comprises between 12 and 100 bases complementary to a RNA molecule of HBV. Even more preferably, a nucleic acid molecule that modulates HBV expression comprises between 14 and 24 bases complementary to a RNA molecule of HBV.

The invention provides a method for producing a class of nucleic acid based gene modulating agents that exhibit a high degree of specificity for the RNA of a desired target. For example, an enzymatic nucleic acid molecule is preferably targeted to a highly conserved sequence region of target RNAs encoding HBV (and specifically a HBV genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., enzymatic nucleic acid molecules and antisense) can be expressed from DNA and/or RNA vectors that are delivered to specific cells.

As used in herein “cell” is used in its usual biological sense, and does not refer to an entire multicellular organism. A cell can, for example, be in vitro, e.g., in cell culture, or present in a multicellular organism, including, e.g., birds, plants, and mammals such as humans, cows, sheep, apes, monkeys, swine, dogs, and cats. The cell can be prokaryotic (e.g., bacterial cell) or eukaryotic (e.g., mammalian or plant cell).

By “HBV proteins” is meant, a peptide or protein comprising a component of HBV and/or encoded by a HBV gene.

By “highly conserved sequence region” is meant, a nucleotide sequence of one or more regions in a target gene that does not vary significantly from one generation to the other or from one biological system to the other.

Nucleic acid-based inhibitors of HBV expression are useful for the prevention and/or treatment of HBV infection, liver failure, hepatocellular carcinoma, or any other disease or conditions that relates to HBV infection.

By “related” is meant that the reduction of HBV expression (and specifically a HBV gene) RNA levels and thus reduction in the level of the respective protein relieves, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers.

In another embodiment, the invention features antisense nucleic acid molecules and 2-5A chimera including sequences complementary to the HBV RNA. Similarly, triplex molecules can be targeted to corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two (or even more) non-contiguous substrate sequences. In addition, two (or even more) non-contiguous sequence portions of an antisense molecule can be complementary to a target sequence.

In preferred embodiments, the invention features a method for the analysis of HBV proteins. This method is useful in determining the efficacy of HBV inhibitors. Specifically, the instant invention features an assay for the analysis of HBsAg proteins and secreted alkaline phosphatase (SEAP) control proteins to determine the efficacy of agents used to modulate HBV expression.

The method consists of coating a micro-titer plate with an antibody such as anti-HBsAg Mab (for example, Biostride B88-95-31ad,ay) at 0.1 to 10 μg/ml in a buffer (for example, carbonate buffer, such as Na₂CO₃ 15 mM, NaHCO₃ 35 mM, pH 9.5) at 4° C. overnight. The microtiter wells are then washed with PBST or the equivalent thereof, (for example, PBS, 0.05% Tween 20) and blocked for 0.1-24 hr at 37° C. with PBST, 1% BSA or the equivalent thereof. Following washing as above, the wells are dried (for example, at 37° C. for 30 min). Biotinylated goat anti-HBsAg or an equivalent antibody (for example, Accurate YVS1807) is diluted (for example at 1:1000) in PBST and incubated in the wells (for example, 1 hr. at 37° C.). The wells are washed with PBST (for example, 4×). A conjugate, (for example, Streptavidin/Alkaline Phosphatase Conjugate, Pierce 21324) is diluted to 10-10,000 ng/ml in PBST, and incubated in the wells (for example, 1 hr. at 37° C.). After washing as above, a substrate (for example, p-nitrophenyl phosphate substrate, Pierce 37620) is added to the wells, which are then incubated (for example, 1 hr. at 37° C.). The optical density is then determined (for example, at 405 nm). SEAP levels are then assayed, for example, using the Great EscAPe® Detection Kit (Clontech K2041-1), as per the manufacturers instructions. In the above example, incubation times and reagent concentrations can be varied to achieve optimum results, a non-limiting example is described in Example 6.

Comparison of this HBsAg ELISA method to a commercially available assay from World Diagnostics, Inc. 15271 NW 60^{th} Ave, #201, Miami Lakes, Fla. 33014 (305) 827-3304 (Cat. No. EL10018) demonstrates an increase in sensitivity (signal:noise) of 3-20 fold.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present that do not interfere with such cleavage. Thus, a core region of an enzymatic nucleic acid molecule can, for example, include one or more loop, stem-loop structure, or linker that does not prevent enzymatic activity. Thus, various regions in the nucleic acid sequence can be such a loop, stem-loop, nucleotide linker, and/or non-nucleotide linker and can be represented generally as sequence “X”. The nucleic acid molecules of the instant invention, such as Hammerhead, Inozyme, G-cleaver, amberzyme, zinzyme, DNAzyme, antisense, 2-5A antisense, triplex forming nucleic acid, and decoy nucleic acids, can contain other sequences or non-nucleotide linkers that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, a non-nucleotide linker X is as defined herein. Non-nucleotides as can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and down-regulate HBV (and specifically a HBV gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, recombinant vectors capable of expressing enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to target RNA and down-regulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that allows for introduction into a desired target cell. Antisense DNA and DNAzymes can be expressed via the use of a single stranded DNA intracellular expression vector.

The term “vectors” as used herein refers to any nucleic acid- and/or viral-based technique used to deliver a desired nucleic acid.

The term “patient” as used herein refers to an organism that is a donor or recipient of explanted cells or the cells themselves. “Patient” also refers to an organism to which the nucleic acid molecules of the invention can be administered. Preferably, a patient is a mammal or mammalian cells. More preferably, a patient is either a human or human cells.

The term “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme, for example with a nucleic acid molecule comprising chemical modifications. In some cases, the activity or stability of the nucleic acid molecule can be decreased (i.e., less than ten-fold), but the overall activity of the nucleic acid molecule is enhanced, in vivo.

Nucleic acid molecules of the instant invention, individually, or in combination or in conjunction with other drugs, can be used to treat diseases or conditions discussed above. For example, to treat a disease or condition associated with the levels of HBV, a patient can be treated, or other appropriate cells can be treated, as is evident to those skilled in the art, individually or in combination with one or more drugs under conditions suitable for the treatment.

In a further embodiment, the described molecules, such as antisense or enzymatic nucleic acid molecules, can be used in combination with other known treatments to treat conditions or diseases discussed above. For example, the described molecules can be used in combination with one or more known therapeutic agents to HBV infection or cancer, for example hepatocellular carcinoma, and any other disease or condition that respond to the modulation of HBV expression.

In a further embodiment, the described molecules, such as antisense or enzymatic nucleic acid molecules, can be used in combination with other known treatments to treat conditions or diseases discussed above. For example, the described molecules could be used in combination with one or more known therapeutic agents to treat HBV infection, hepatitis, hepatocellular carcinoma, cancer, cirrhosis, and liver failure. Such therapeutic agents can include, but are not limited to nucleoside analogs selected from the group comprising Lamivudine (3TC®), L-FMAU, and/or adefovir dipivoxil (for a review of applicable nucleoside analogs, see Colacino and Staschke, 1998, Progress in Drug Research, 50, 259-322). Immunomodulators selected from the group comprising Type 1 Interferon, such as in the method of claim 47, wherein said type I interferon is interferon alpha, interferon beta, consensus interferon, polyethylene glycol interferon, polyethylene glycol interferon alpha 2a, polyethylene glycol interferon alpha 2b, or polyethylene glycol consensus interferon, and therapeutic vaccines, steroids, and 2′-5′ oligoadenylates (for a review of 2′-5′ Oligoadenylates, see Charubala and Pfleiderer, 1994, Progress in Molecular and Subcellular Biology, 14, 113-138).

In another embodiment, the invention features nucleic acid-based inhibitors (e.g., enzymatic nucleic acid molecules, antisense nucleic acids, 2-5A antisense chimeras, triplex DNA, antisense nucleic acids containing RNA cleaving chemical groups) and methods for their use to down regulate or inhibit the expression of genes (e.g., HBV) capable of progression and/or maintenance of cancer and/or other disease states that respond to the modulation of HBV expression.

Other features and advantages of the invention will be apparent from the following description of the preferred embodiments thereof, and from the claims.

### DETAILED DESCRIPTION OF THE INVENTION

First the drawings will be described briefly.

### DRAWINGS

FIG. 1 shows examples of chemically stabilized enzymatic nucleic acid motifs. HH Rz, represents hammerhead ribozyme motif (Usman et al., 1996, Curr. Op. Struct. Bio., 1, 527); NCH Rz represents NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

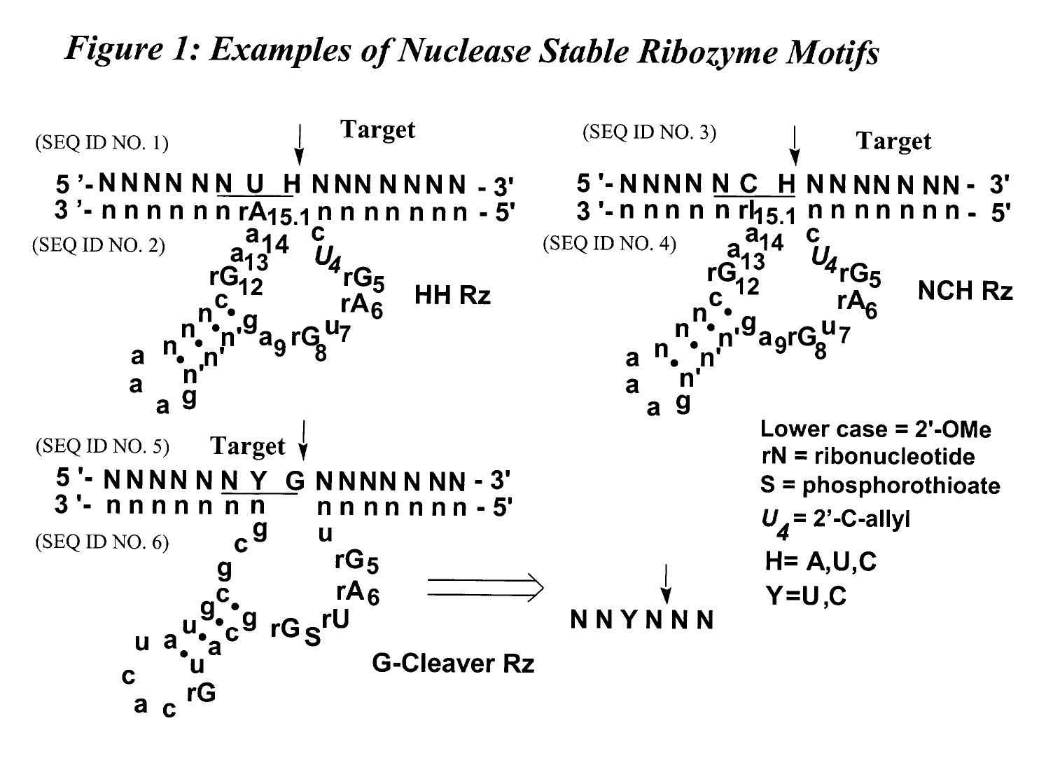

FIG. 2 shows an example of the Amberzyme enzymatic nucleic acid motif that is chemically stabilized (see for example Beigelman et al., International PCT publication No. WO 99/55857).

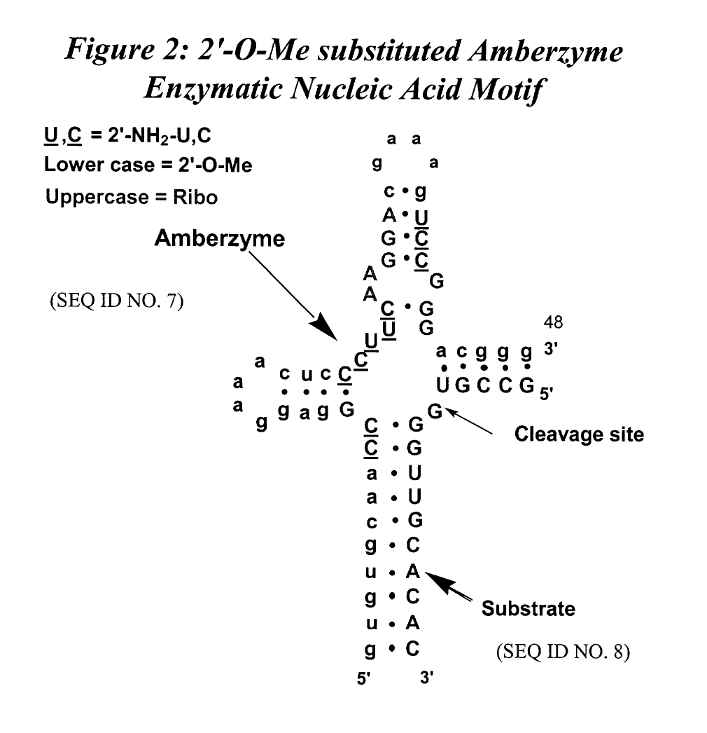

FIG. 3 shows an example of the Zinzyme enzymatic nucleic acid motif that is chemically stabilized (see for example Beigelman et al., Beigelman et al., International PCT publication No. WO 99/55857).

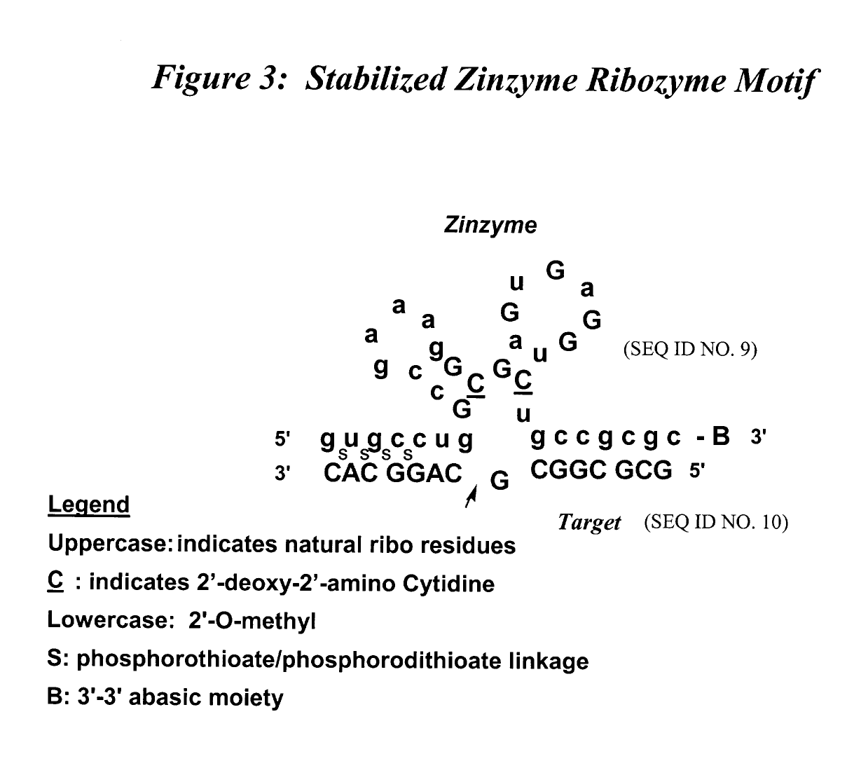

FIG. 4 shows an example of a DNAzyme motif (e.g., “10-23”) described by Santoro et al., 1997, PNAS, 94, 4262.

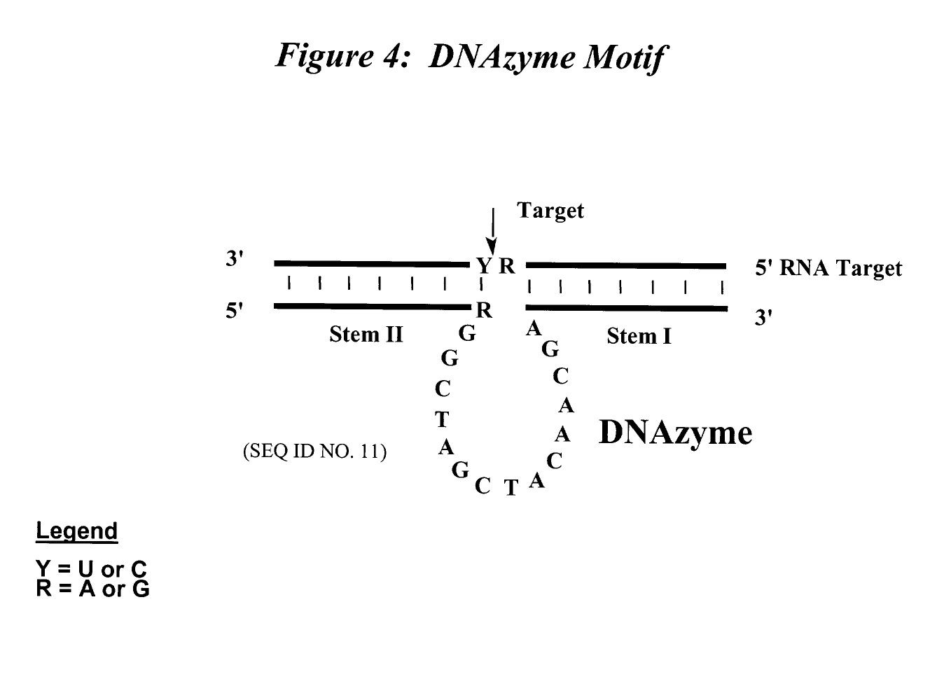

FIG. 5 shows a graph depicting HepG2.2.15 tumor growth in athymic nu/nu female mice as tumor volume (mm³) vs time (days).

FIG. 6 shows a graph depicting HepG2.2.15 tumor growth in athymic nu/nu female mice as tumor volume (mm³) vs time (days). Inoculated HepG2.2.15 cells were selected for antibiotic resistance to G418 before introduction into the mouse.

### MECHANISM OF ACTION OF NUCLEIC ACID MOLECULES OF THE INVENTION AS IS KNOW IN THE ART

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174, filed on Sep. 21, 1998). All of these references are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra)

2′-5′ Oligoadenylates: The 2-5 A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme. The covalent attachment of 2′-5′ oligoadenylate structures is not limited to antisense applications, and can be further elaborated to include attachment to nucleic acid molecules of the instant invention.

Enzymatic Nucleic Acid: Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions. Nucleic acid molecules of this invention will block to some extent HBV protein expression and can be used to treat disease or diagnose disease associated with the levels of HBV.

The enzymatic nature of an enzymatic nucleic acid molecule can allow the concentration of enzymatic nucleic acid molecule necessary to affect a therapeutic treatment to be lower than a nucleic acid molecule lacking enzymatic activity, such as an antisense nucleic acid. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to down-regulate HBV expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant HBV protein, wild-type HBV protein, mutant HBV RNA, wild-type HBV RNA, other proteins and/or RNAs involved in HBV activity, compounds, metals, polymers, molecules and/or drugs that are targeted to HBV expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type HBV, mutant HBV, a component of HBV, and/or a predetermined cellular component that modulates HBV activity. In a specific example, allosteric enzymatic nucleic acid molecules that are activated by interaction with a RNA encoding HBV protein are used as therapeutic agents in vivo. The presence of RNA encoding the HBV protein activates the allosteric enzymatic nucleic acid molecule that subsequently cleaves the RNA encoding HBV protein resulting in the inhibition of HBV protein expression. In this manner, cells that express the HBV protein are selectively targeted.

In another non-limiting example, an allozyme can be activated by a HBV protein, peptide, or mutant polypeptide that caused the allozyme to inhibit the expression of HBV gene, by, for example, cleaving RNA encoded by HBV gene. In this non-limiting example, the allozyme acts as a decoy to inhibit the function of HBV and also inhibit the expression of HBV once activated by the HBV protein.

The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, provided below are specific examples of such methods, not limiting to those in the art. Enzymatic nucleic acid molecules to such targets are designed as described in those applications and synthesized to be tested in vitro and in vivo, as also described. The sequences of human HBV RNAs were screened for optimal enzymatic nucleic acid target sites using a computer-folding algorithm. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

Enzymatic nucleic acid molecule binding/cleavage sites are identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Enzymatic nucleic acid molecule binding/cleavage sites are identified and are designed to anneal to various sites in the RNA target. The binding arms are complementary to the target site sequences described above. The nucleic acid molecules are chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic acid Molecules

Synthesis of nucleic acids greater than 100 nucleotides in length can be difficult using automated methods, and the therapeutic cost of such molecules can be prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or NCH enzymatic nucleic acid molecules) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20 ° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA and chemically modified RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead enzymatic nucleic acid molecules or binding attenuated control (BAC) oligonucleotides can be synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Enzymatic nucleic acid molecules are purified by gel electrophoresis using general methods or are purified by high pressure liquid chromatography (HPLC; See Wincott et al., Supra, the totality of which is hereby incorporated herein by reference) and are re-suspended in water.

Optimizing Activity of the Nucleic Acid Molecule of the Invention

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into enzymatic nucleic acid molecules without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid molecules are also generally more resistant to nucleases than unmodified nucleic acid molecules. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al, 1992, Methods in Enzymology 211,3-19 (incorporated by reference herein)) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

The term “cap structure” as used herein refers to chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The term “non-nucleotide” as used herein refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example methoxyethyl or ethoxymethyl.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

The term “nucleotide” as used herein refers to a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein. There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1 -methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “nucleoside” as used herein refers to a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, form acetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmacker et al, 1994, Novel Backbone Replacements for Oligonucleotides, in Carbohydrate Modifications in Antisense Research, ACS, 24-39. These references are hereby incorporated by reference herein.

The term “abasic” as used herein refers to sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

The term “unmodified nucleoside” as used herein refers to one of the bases adenine, cytosine, guanine, thymine, uracil joined to the 1′ carbon of β-D-ribo-furanose.

The term “modified nucleoside” as used herein refers to any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and enzymatic nucleic acid molecule) structure can be made to enhance the utility of these molecules. For example, such modifications can enhance shelf life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Compounds and Therapies of the Invention

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819, all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

The term “systemic administration” as used herein refers to in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes, which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, anti oxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; Skillern et al., International PCT Publication No. WO 00/22113; Conrad, International PCT Publication No. WO 00/22114; and Conrad, U.S. Pat. No. 6,054,299; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, nucleic acid molecules of the present invention are preferably expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors are preferably DNA plasmids or viral vectors. Enzymatic nucleic acid molecule expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the patient followed by reintroduction into the patient, or by any other means that would allow for introduction into the desired target cell (for a review see Couture et al., 1996, TIG., 12, 510).

One aspect of the invention features an expression vector comprising a nucleic acid sequence encoding at least one of the nucleic acid molecules of the instant invention. The nucleic acid sequence encoding the nucleic acid molecule of the instant invention is operable linked in a manner that allows expression of that nucleic acid molecule.

In another aspect, the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner that allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res.., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as enzymatic nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as enzymatic nucleic acid molecules in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above enzymatic nucleic acid molecule transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

Another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner that allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, Hammerhead, DNAzyme, NCH, Amberzyme, Zinzyme or G-Cleaver enzymatic nucleic acid molecule and binding/cleavage sites within HBV RNA.

### Example 1

Identification of Potential Target Sites in Human HBV RNA

The sequence of human HBV was screened for accessible sites using a computer-folding algorithm. Regions of the RNA that did not form secondary folding structures and contained potential enzymatic nucleic acid molecule and/or antisense binding/cleavage sites were identified.

### Example 2

Selection of Enzymatic Nucleic Acid Cleavage Sites in Human HBV RNA

Enzymatic nucleic acid molecule target sites were chosen by analyzing sequences of Human HBV (accession number: AF100308.1) and prioritizing the sites on the basis of folding. Enzymatic nucleic acid molecules were designed that could bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core were eliminated from consideration. As noted herein, varying binding arm lengths can be chosen to optimize activity. Generally, at least 5 bases on each arm are able to bind to, or otherwise interact with, the target RNA.

### Example 3

Chemical Synthesis and Purification of Enzymatic Nucleic Acid Molecules and Antisense for Efficient Cleavage and/or Blocking of HBV RNA

Enzymatic nucleic acid molecules and antisense constructs were designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs were also synthesized from DNA templates using bacteriophage T7 RNA polymerase (Milligan and Uhlenbeck, 1989, Methods Enzymol. 180, 51). Enzymatic nucleic acid molecules and antisense constructs were purified by gel electrophoresis using general methods or were purified by high pressure liquid chromatography (HPLC; see Wincott et al., supra; the totality of which is hereby incorporated herein by reference) and were resuspended in water.

### Example 4

Enzymatic Nucleic Acid Molecule Cleavage of HBV RNA Target in vitro

Enzymatic nucleic acid molecules targeted to the human HBV RNA are designed and synthesized as described above. These enzymatic nucleic acid molecules can be tested for cleavage activity in vitro, for example using the following procedure.

Cleavage Reactions: Full-length or partially full-length, internally-labeled target RNA for enzymatic nucleic acid molecule cleavage assay is prepared by in vitro transcription in the presence of [α-³²p] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

Analysis of HBV DNA Expression a HepG2.2.15 Murine Model

The development of new antiviral agents for the treatment of chronic Hepatitis B has been aided by the use of animal models that are permissive to replication of related Hepadnaviridae such as Woodchuck Hepatitis Virus (WHV) and Duck Hepatitis Virus (DHV). In addition the use of transgenic mice have also been employed. The human hepatoblastoma cell line, HepG2.2.15, implanted as a subcutaneous (SC) tumor, was evaluated in terms of its usefulness in producing Hepatitis B viremia in mice. This model is useful for evaluating new HBV therapies. The study showed that in mice bearing HepG2.2.15 SC tumors, HBV viremia was present. HBV DNA was detected in serum beginning on Day 35. Maximum serum viral levels reached 1.9×10⁵ copies/mL by day 49. This study also determined that the minimum tumor volume associated with viremia was 300 mm³. Therefore, the HepG2.2.15 cell line grown as a SC tumor produces a useful model of HBV viremia in mice. This new model can be suitable for evaluating new therapeutic regimens for chronic Hepatitis B.

HepG2.2.15 tumor cells contain a slightly truncated version of viral HBV DNA and sheds HBV particles. The purpose of this study was to identify what time period viral particles are shed from the tumor. Serum was analyzed for presence of HBV DNA over a time course after HepG2.2.15 tumor inoculation in Athymic Ncr nu/nu mice.

Experiment 1

HepG2.2.15 cells were carried and expanded in DMEM/10% FBS/2.4%HEPES/1%NEAA/1% Glutamine/1% Sodium Pyruvate media. Cells were resuspended in Dulbecco's PBS with calcium/magnesium for injection. One hundred microliters of the tumor cell suspension (at a concentration of 1×108 cells/mL) were injected subcutaneously in the flank of NCR nu/nu female mice with a 23g1 needle and 1 cc syringe, thereby giving each mouse 1×10⁷ cells. Tumors were allowed to grow for a period of up to 49 days post tumor cell inoculation. Serum was sampled for analysis on days 1, 7, 14, 35, 42 and 49 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Results

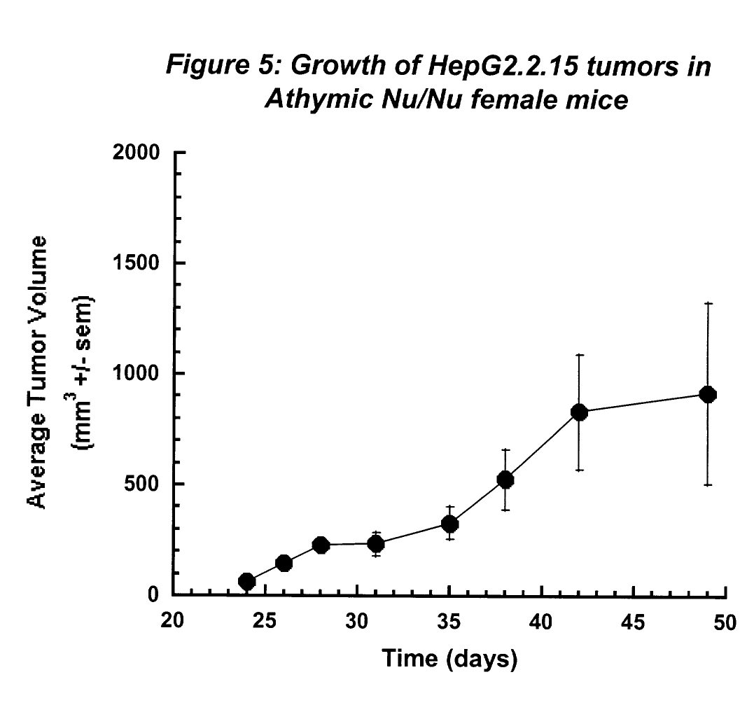

When athymic nu/nu female mice are subcutaneously injected with HepG2.2.15 cells and form tumors, HBV DNA is detected in serum (peak serum level was 1.9×10⁵ copies/mL). There is a positive correlation (rs=0.7, p<0.01) between tumor weight (milligrams) and HB viral copies/mL serum. FIG. 5 shows a plot of HepG2.2.15 tumors in nu/nu female mice as tumor volume vs time. Table III shows the concentration of HBV DNA in relation to tumor size in the HepG2.2.15 implanted nu/nu female mice used in the study.

Experiment 2

HepG2.2.15 cells were carried and expanded in DMEM/10% FBS/2.4%HEPES/1%NEAA/1% Glutamine/1% Sodium Pyruvate media containing 400 μg/ml G418 antibiotic. G418-resistant cells were resuspended in Dulbecco's PBS with calcium/magnesium for injection. One hundred microliters of the tumor cell suspension (at a concentration of 1×108 cells/mL) were injected subcutaneously in the flank of NCR nu/nu female mice with a 23g1 needle and 1 cc syringe, thereby giving each mouse 1×10⁷ cells. Tumors were allowed to grow for a period of up to 49 days post tumor cell inoculation. Serum was sampled for analysis on day 37 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Results

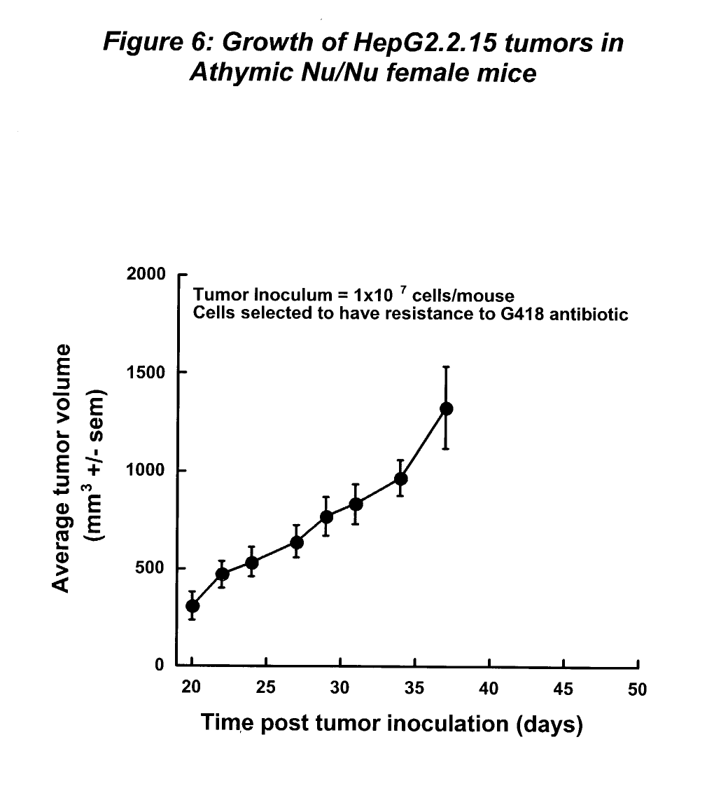

When athymic nu/nu female mice are subcutaneously injected with G418 antibiotic resistant HepG2.2.15 cells and form tumors, HBV DNA is detected in serum (peak serum level was 4.0×10⁵ copies/mL). There is a positive correlation (rs=0.7, p<0.01) between tumor weight (milligrams) and HB viral copies/mL serum. FIG. 6 shows a plot of HepG2.2.15 tumors in nu/nu female mice as tumor volume vs time. Table IV shows the concentration of HBV DNA in relation to tumor size in the G418 antibiotic resistant HepG2.2.15 implanted nu/nu female mice used in the study.

Cell Culture Models

As previously mentioned, HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA is co-transfected with enzymatic nucleic acid molecules in cell culture. Such an approach has been used to report intracellular enzymatic nucleic acid molecule activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV. In these cells only enzymatic nucleic acid molecule is delivered; however, a delivery screen must be performed. Intracellular HBV gene expression is assayed using known methods, for example, by a Taqman® assay for HBV RNA, or by ELISA for HBV protein. Extracellular virus is assayed by, for example, PCR for DNA or ELISA for protein. Antibodies are commercially available for HBV surface antigen and core protein. A secreted alkaline phosphatase expression plasmid can be used to normalize for differences in transfection efficiency and sample recovery.

Animal Models

There are several small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Guidotti et al, 1995, J. Virology, 69, 10, 6158-6169; Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to test a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

Indications

Particular degenerative and disease states that can be associated with HBV expression modulation include but are not limited to, HBV infection, hepatitis, cancer, tumorigenesis, cirrhosis, liver failure and others.

The present body of knowledge in HBV research indicates the need for methods to assay HBV activity and for compounds that can regulate HBV expression for research, diagnostic, and therapeutic use.

Lamivudine (3TC®), L-FMAU, adefovir dipivoxil, type 1 Interferon, PEG Interferons, therapeutic vaccines, steroids, and 2′-5′ Oligoadenylates are non-limiting examples of pharmaceutical agents that can be combined with or used in conjunction with the nucleic acid molecules (e.g. enzymatic nucleic acid molecules and antisense molecules) of the instant invention. Those skilled in the art will recognize that other drugs or other therapies can similarly and readily be combined with the nucleic acid molecules of the instant invention (e.g. enzymatic nucleic acid molecules and antisense molecules) and are, therefore, within the scope of the instant invention.

Diagnostic Uses

The nucleic acid molecules of this invention (e.g., ribozymes) can be used as diagnostic tools to examine genetic drift and mutations within diseased cells or to detect the presence of HBV RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These assays can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HBV-related condition. Such RNA is detected by determining the presence of a cleavage product after treatment with an enzymatic nucleic acid molecule using standard methodology.

In a specific example, enzymatic nucleic acid molecules which cleaves only wild-type or mutant forms of the target RNA are used for the assay. The first enzymatic nucleic acid molecule identifys wild-type RNA present in the sample and the second enzymatic nucleic acid molecule identifys mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates can also serve to generate size markers for the analysis of wild-type and mutant RNAs in the sample population. Thus each analysis involves two enzymatic nucleic acid molecules, two substrates and one unknown sample which are combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HBV) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention.

Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring enzymatic nucleic acid molecules

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage products with
3′-OH and 5′-guanosine.
Additional protein cofactors required in some cases to help folding and maintenance of
the active structure.
Over 300 known members of this class. Found as an intervening sequence in Tetrahymena
thermophila rRNA, fungal mitochondria, chloroplasts, phage T4, blue-green algae, and
others.
Major structural features largely established through phylogenetic comparisons,
mutagenesis, and biochemical studies [ⁱ,ⁱⁱ].
Complete kinetic framework established for one ribozyme [ⁱⁱⁱ, ^{iv}, ^{v}, ^{vi}].
Studies of ribozyme folding and substrate docking underway [^{vii}, ^{viii}, ^{ix}].
Chemical modification investigation of important residues well established [^{x}, ^{xi}].
The small (4-6 nt) binding site can make this ribozyme too non-specific for targeted RNA
cleavage, however, the Tetrahymena group I intron has been used to repair a “defective”
β-galactosidase message by the ligation of new β-galactosidase sequences onto the
defective message [^{xii}].
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage products with 3′-
OH and 5′-phosphate.
RNAse P is found throughout the prokaryotes and eukaryotes. The RNA subunit has
been sequenced from bacteria, yeast, rodents, and primates.
Recruitment of endogenous RNAse P for therapeutic applications is possible through
hybridization of an External Guide Sequence (EGS) to the target RNA [^{xiv}, ^{xv}]
Important phosphate and 2′ OH contacts recently identified [^{xvi}, ^{xvii}]
Group II Introns
Size: >1000 nucleotides.
Trans cleavage of target RNAs recently demonstrated [^{xviii}, ^{xix}].
Sequence requirements not fully determined.
Reaction mechanism: 2′-OH of an internal adenosine generates cleavage products with 3′-
OH and a “lariat” RNA containing a 3′-5′ and a 2′-5′ branch point.
Only natural ribozyme with demonstrated participation in DNA cleavage [^{xx}, ^{xxi}] in
addition to RNA cleavage and ligation.
Major structural features largely established through phylogenetic comparisons [^{xxii}].
Important 2′ OH contacts beginning to be identified [^{xxiii}]
Kinetic framework under development [^{xxiv}]
Neurospora VS RNA
Size: ˜444 nucleotides.
Trans cleavage of hairpin target RNAs recently demonstrated [^{xxv}].
Sequence requirements not fully determined.
Reaction mechanism: attack by 2′-OH 5′ to the scissile bond to generate cleavage products
with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage products
with 2′,3′-cyclic phosphate and 5′-OH ends.
14 known members of this class. Found in a number of plant pathogens (virusoids) that
use RNA as the infectious agent.
Essential structural features largely defined, including 2 crystal structures [^{xxvi}, ^{xxvii}]
Minimal ligation activity demonstrated (for engineering through in vitro selection) [^{xxviii}]
Complete kinetic framework established for two or more ribozymes [^{xxix}].
Chemical modification investigation of important residues well established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable number to the 3′-side
of the cleavage site.
Reaction mechanism: attack by 2′-OH 5′ to the scissile bond to generate cleavage products
with 2′,3′-cyclic phosphate and 5′-OH ends.
3 known members of this class. Found in three plant pathogen (satellite RNAs of the
tobacco ringspot virus, arabis mosaic virus and chicory yellow mottle virus) which uses
RNA as the infectious agent.
Essential structural features largely defined [^{xxxi}, ^{xxxii}, ^{xxxiii}, ^{xxxiv}]
Ligation activity (in addition to cleavage activity) makes ribozyme amenable to
engineering through in vitro selection [^{xxxv}]
Complete kinetic framework established for one ribozyme [^{xxxvi}].
Chemical modification investigation of important residues begun [^{xxxvii}, ^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no sequences 5′
of cleavage site are required. Folded ribozyme contains a pseudoknot structure [^{xl}].
Reaction mechanism: attack by 2′-OH 5′ to the scissile bond to generate cleavage products
with 2′,3′-cyclic phosphate and 5′-OH ends.
Only 2 known members of this class. Found in human HDV.
^{xli}Circular form of HDV is active and shows increased nuclease stability [^{xlii}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H.. The time dependence of chemical modification reveals slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at the Tetrahymena ribozyme reaction site. Science (Washington, D. C.) (1995), 267(5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5-Splice Site Selection and Transition State Stabilization. Biochemistry (1996), 35(4), 1201-11.
^{xii}Sullenger, Bruce A.; Cech, Thomas R.. Ribozyme-mediated repair of defective mRNA by targeted trans-splicing. Nature (London) (1994), 371 (6498), 619-22.
^{xiii}Robertson, H. D.; Altman, S,; Smith, J. D. J. Biol. Chem 247, 5243-5251 (1972).
^{xiv}Forster, Anthony C.; Altman, Sidney. External guide sequences for an RNA enzyme. Science (Washington, D. C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R.. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2-hydroxyl groups within a group II intron active site. Science (Washington, D. C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. I.; Collins, Richard A.. Efficient trans-cleavage of a stem-loop RNA substrate by a ribozyme derived from Neurospora VS RNA. EMBO J. (1995), 14(2), 368-76.
^{xxvi}Scoff, W. G., Finch, J. T., Aaron, K. The crystal structure of an all RNA hammerhead ribozyme: Aproposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx}Beigelman, L., et al., Chemical modifications of hammerhead ribozymes. I. Biol. Chem., (1995) 270, 25702-25708.
^{xxxi}Hampel, Arnold; Tritz, Richard; Hicks, Margaret Cruz, Phillip. ‘Hairpin’ catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl}Perrotta, Anne T.; Been, Michael D.. A pseudoknot-like structure required for efficient self-cleavage of hepatitis delta virus RNA. Nature (London) (1991), 350(6317), 434-6.
^{xli}
^{xlii}Puttaraju, M.; Perrotta, Anne T.; Been, Michael D.. A circular trans-acting hepatitis delta virus ribozyme. Nucleic Acids Res. (1993), 21(18), 4253-8.

TABLE II

| | | | | | |
|---|---|---|---|---|---|
| Reagent | Equivalents | Amount | Wait Time* DNA | Wait Time* 2′-O-methyl | Wait Time* RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents: DNA/ | Amount: DNA/2′-O- | | Wait Time* 2′-O- | |
|---|---|---|---|---|---|
| Reagent | 2′-O-methyl/Ribo | methyl/Ribo | Wait Time* DNA | methyl | Wait Time* Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE III
Comparison of Tumor Weight to HBV DNA concentration in mice
inoculated with HepG2.2.15 cells

| Time point | HBV DNA | Tumor weight |
|---|---|---|
| (days) | copies/mL serum | (milligrams) |
| 1 | Below detection | No tumor |
| 1 | Below detection | No tumor |
| 1 | Below detection | No tumor |
| 1 | Below detection | No tumor |
| 7 | Below detection | No tumor |
| 7 | Below detection | No tumor |
| 7 | Below detection | No tumor |
| 7 | Below detection | No tumor |
| 14 | Below detection | No tumor |
| 14 | Below detection | No tumor |
| 14 | Below detection | No tumor |
| 14 | Below detection | No tumor |
| 35 | 356 | 33 |
| 35 | 125083 | 167 |
| 35 | 578 | No tumor |
| 35 | 386 | 56 |
| 42 | 493 | No tumor |
| 42 | 114431 | 790 |
| 42 | 94025 | 359 |
| 42 | 111882 | 647 |
| 49 | 189885 | 816 |
| 49 | Below detection | No tumor |
| 49 | 293 | 90 |
| 49 | 41477 | 2521 |

TABLE IV
Comparison of Tumor Weight to HBV DNA concentration in mice
inoculated with G418 resistant HepG2.2.15 cells

| Time point | HBV DNA copies/mL | Tumor weight |
|---|---|---|
| (days) | serum | (milligrams) |
| 37 | 7000 | 1120.0 |
| 37 | no sample | no sample |
| 37 | 400000 | 1962.3 |
| 37 | 26000 | 558.5 |
| 37 | 380000 | 2286.0 |
| 37 | 100 | 317.2 |
| 37 | 52000 | 1429.0 |
| 37 | 100 | 427.4 |
| 37 | 26000 | 813.2 |
| 37 | 1400 | 631.6 |
| 37 | 186000 | 1101.5 |
| 37 | 134000 | 1573.0 |
| 37 | 17800 | 1040.0 |
| 37 | 16600 | 1327.2 |
| 37 | 8200 | 275.7 |
| 37 | 68000 | 632.8 |
| 37 | 24000 | 1090.0 |
| 37 | 58000 | 1082.7 |
| 37 | 12400 | 1116.3 |
| 37 | 100 | 763.3 |

What we claim is:

1. A mouse implanted with HepG2.2.15 cells, wherein said mouse sustains the propagation of HEPG2.2.15 cells and HBV production.

2. The mouse of claim 1, wherein said mouse has been infected with HBV for at least one week.

3. The mouse of claim 1, wherein said mouse has been infected with HBV for at least four weeks.

4. The mouse of claim 1, wherein said mouse has been infected with HBV for at least eight weeks

5. The mouse of claim 1, wherein said mouse is an immunocompromised mouse.

6. The mouse of claim 5, wherein said mouse is a nu/nu mouse.

7. The mouse of claim 5, wherein said mouse is a scid/scid mouse.

8. A method of producing a mouse according to claim 1 comprising injecting HepG2.2.15 cells into said mouse under conditions suitable for the propagation of the HepG2.2.1.5 cells in said mouse.

9. The method of claim 8, wherein said mouse is a nu/nu mouse.

10. The method of claim 8, wherein said mouse is a scid/scid mouse.

11. The method of claim 8, wherein said injection is subcutaneous injection.

12. The method of claim 8, wherein said HepG2.2.15 cells are suspended in Dulbecco's PBS solution including calcium and magnesium.

13. A method for screening a compound or compounds for anti-HBV activity comprising administering said compound(s) to a mouse of claim 1 and monitoring the level of HBV DNA to determine anti-HBV activity.

14. The method of claim 13, wherein said compound is a nucleic acid molecule, administered alone or in combination with another compound.

15. The method of claim 14, wherein said nucleic acid molecule is an enzymatic nucleic acid molecule.

16. The method of claim 14, wherein said nucleic acid molecule is an antisense nucleic acid molecule.

17. The method of claim 14, wherein said other compound is Lamivudine.

18. The method of claim 14, wherein said other compound is interferon.

19. An immunocompromised non-human mammal implanted with HepG2.2.15 cells, wherein said non-human mammal is susceptible to HBV infection and capable of sustaining HBV DNA expression.

20. The method of claim 14, wherein said other compound is a second nucleic acid molecule which is different from the first nucleic acid molecule.
